(19) United States
(12) Plant Patent Application Publication
(10) Pub. No.: US 2019/0223365 P1
(43) Pub. Date: Jul. 18, 2019

(54) HELLEBORUS PLANT NAMED 'JHE00202'

(50) ;

(71) Applicant: Thierry van Paemel, Oostkamp (BE)

(72) Inventor: Thierry van Paemel, Oostkamp (BE)

(73) Assignee: FLOREAC N.V. div MICROFLOR, Lochristi (BE)

(21) Appl. No.: 15/732,943

(22) Filed: Jan. 12, 2018

(51) Int. Cl. A01H 6/00

(52) U.S. Cl. PLT/439

(57) ABSTRACT

A new and distinct cultivar of Helleborus plant named ‘JHE00202’, characterized by its compact, upright and mounding plant habit; moderately vigorous growth habit and moderate growth rate; dark green-colored leaves; early and freely flowering habit; single light yellow green-colored flowers; and good garden performance.

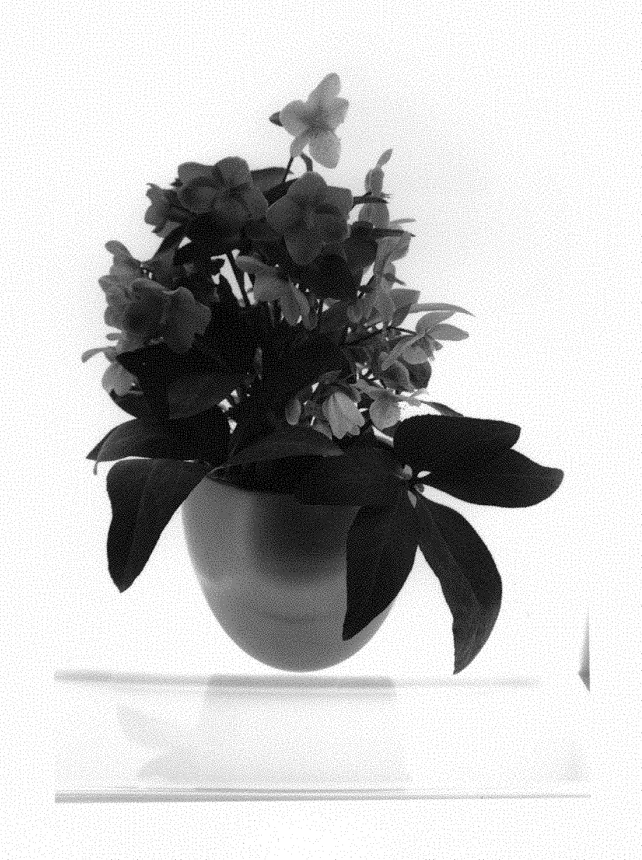

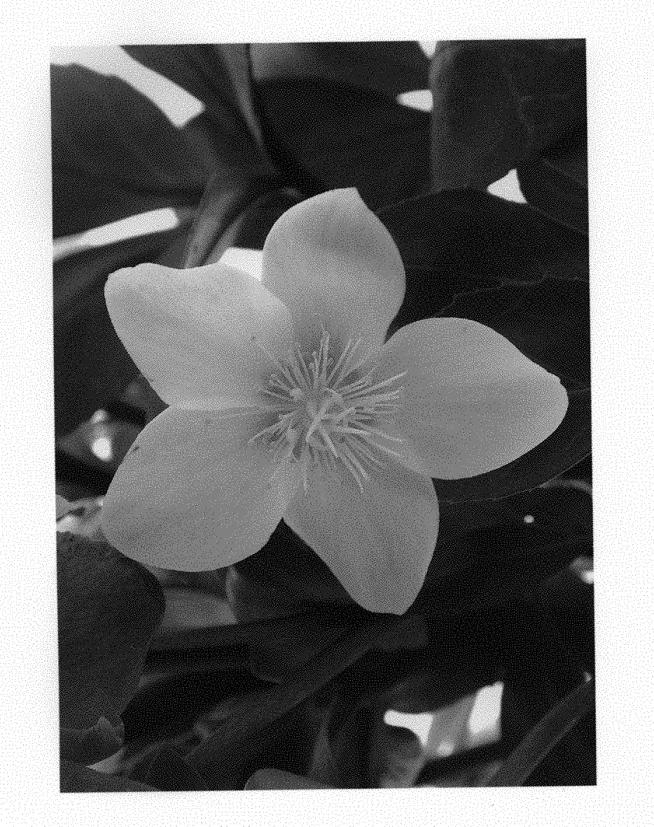

Botanical designation: Helleborus X ericsmithii.

Cultivar denomination: ‘JHE00202’.

### BACKGROUND OF THE INVENTION

The present invention relates to a new and distinct cultivar of Helleborus plant, botanically known as Helleborus X ericsmithii and hereinafter referred to by the name ‘JHE00202’. Helleborus X ericsmithii is a hybrid between Helleborus niger and Helleborus X sternii.

The new Helleborus plant is a product of a planned breeding program conducted by the Inventor in Oostkamp, Belgium. The objective of the breeding program was to create new early-flowering Helleborus plants with attractive plant habit and flowers.

The new Helleborus plant originated from a cross-pollination conducted by the Inventor in Oostkamp, Belgium in 2011 of a proprietary selection of Helleborus niger identified as code number 6032, not patented, as the female, or seed, parent with Helleborus X sternii ‘Flame’, not patented, as the male, or pollen, parent. The new Helleborus plant was discovered and selected by the Inventor as a single flowering plant from within the progeny of the stated cross-pollination grown in a controlled greenhouse environment in Oostkamp, Belgium in November, 2014.

Asexual reproduction of the new Helleborus plant by in vitro meristem propagation in a controlled environment in Lochristi, Belgium since August, 2016 has shown that the unique features of this new Helleborus plant are stable and reproduced true to type in successive generations.

### SUMMARY OF THE INVENTION

Plants of the new Helleborus have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘JHE00202’. These characteristics in combination distinguish ‘JHE00202’ as a new and distinct Helleborus plant:
1. Compact, upright and mounding plant habit.
2. Moderately vigorous growth habit and moderate growth rate.
3. Dark green-colored leaves.
4. Early and freely flowering habit.
5. Single light yellow green-colored flowers.
6. Good garden performance.

Plants of the new Helleborus differ primarily from plants of the female parent selection in flower color as plants of the new Helleborus have pure white-colored flowers.

Plants of the new Helleborus differ primarily from plants of the male parent, ‘Flame’, in plant habit as plants of the new Helleborus are more compact and denser than plants of ‘Flame’.

Plants of the new Helleborus can be compared to plants of Helleborus X ericsmithii (Helleborus niger X Helleborus sternii) ‘COSEH 860’, disclosed in U.S. Plant Pat. No. 23,773. In side-by-side comparisons, plants of the new Helleborus differ primarily from plants of ‘COSEH 860’ in the following characteristics:
1. Plants of the new Helleborus flower about one month earlier than plants of ‘COSEH 860’.
2. Leaves of plants of the new Helleborus are not as serrated as leaves of plants of ‘COSEH 860’.

Plants of the new Helleborus can also be compared to plants of Helleborus X nigercors ‘Candy Love’, disclosed in U.S. Plant Pat. No. 19,413. In side-by-side comparisons, plants of the new Helleborus differ primarily from plants of ‘Candy Love’ in the following characteristics:
1. Plants of the new Helleborus are more compact and denser than plants of ‘Candy Love’.
2. Plants of the new Helleborus flower about two to three months earlier than plants of ‘Candy Love’.
3. Leaves of plants of the new Helleborus are not as serrated as leaves of plants of ‘Candy Love’.

### BRIEF DESCRIPTION OF THE PHOTOGRAPHS

The accompanying colored photographs illustrate the overall appearance of the new Helleborus plant showing the colors as true as it is reasonably possible to obtain in colored reproductions of this type. Colors in the photographs may differ slightly from the color values cited in the detailed botanical description which accurately describe the colors of the new Helleborus plant.

The photograph on the first sheet comprises a side perspective view of a typical flowering plant of ‘JHE00202’ grown in a container.

The photograph on the second sheet is a close-up view of a typical flower of ‘JHE00202’.

### DETAILED BOTANICAL DESCRIPTION

The aforementioned photographs and following observations, measurements and values describe plants grown during the autumn and early winter in 17-cm containers in a shadehouse in Lochristi, Belgium and under cultural practices typical of commercial Helleborus production. During the production of the plants, day temperatures ranged from 5° C. to 26° C. and night temperatures ranged from 5° C. to 18° C. Plants were 15 months old when the photographs and the description were taken. In the following description, color references are made to The Royal Horticultural Society Colour Chart, 2015 Edition, except where general terms of ordinary dictionary significance are used.
Botanical classification: Helleborus X ericsmithii ‘JHE00202’.
Parentage:
   Female, or seed, parent.—Proprietary selection of Helleborus niger identified as code number 6032, not patented.
   Male, or pollen, parent.—Helleborus X sternii ‘Flame’, not patented.
Propagation:
   Type.—By in vitro meristem propagation.
   Time to initiate roots, summer.—About six weeks at temperatures about 20° C.
   Time to produce a rooted young plant, summer.—About seven months at temperatures about 20° C.
   Root description.—Medium in thickness, fleshy; typically brown with white-colored root tips, actual color of the roots is dependent on substrate composition, water quality, fertilizer type and formulation, substrate temperature and physiological age of roots.
   Rooting habit.—Moderately freely branching; medium density.
Plant description:
   Plant and growth habit.—Herbaceous perennial; compact, upright and mounding plant habit with flowers held within and above the foliar plane; plant shape is roughly flattened globular; moderately vigorous growth habit and moderate growth rate.
   Plant height, soil level to top of foliar plane.—About 17.2 cm.
   Plant height, soil level to top of floral plane.—About 33.6 cm.
   Plant diameter (area of spread).—About 50.8 cm.
Leaf description:
   Arrangement.—Leaves arranged in a basal rosette; leaves palmately compound with five leaflets per leaf.
   Leaf length.—About 18.7 cm.
   Leaf width.—About 19.3 cm.
   Leaflet length.—About 10.7 cm to 15.5 cm.
   Leaflet width.—About 4.7 cm to 5.6 cm.
   Leaf shape.—Palmate; broadly ovate to orbicular in outline.
   Leaflet shape.—Ovate to narrowly ovate, occasionally elliptical.
   Leaflet apex.—Acute.
   Leaflet base.—Attenuate.
   Leaflet margin.—Slightly serrate to almost entire.
   Leaflet texture and luster, upper surface.—Smooth, glabrous; leathery; moderately glossy.
   Leaflet texture and luster, lower surface.—Smooth, glabrous; leathery; slightly glossy.
   Leaflet venation pattern.—Pinnate.
   Leaflet color.—Developing leaves, upper surface: Close to between 139A and 147A; venation, similar to lamina except for proximally, tinged with close to 187A. Developing leaves, lower surface: Close to 147B; venation, close to N199A. Fully developed leaves, upper surface: Close to between 147A and N189A; venation, close to 143A. Fully developed leaves, lower surface: Close to between 147B and 191A; venation, close to N199A.
   Petioles.—Length: About 12.5 cm. Diameter: About 4.5 mm. Strength: Strong. Texture and luster, upper and lower surfaces: Smooth, glabrous; moderately glossy. Color, upper and lower surfaces: Close to between 146A and 147B; fine and dense dots, close to N199A.
Flower description:
   Flower shape and habit.—Single rotate flowers in terminal cymes; freely and uniform flowering habit with about eight flowers per inflorescence and about 50 flower buds and flowers developing per plant; flowers facing outwardly to slightly nodding.
   Fragrance.—None detected.
   Natural flowering season.—Plants begin flowering about ten months after planting; plants flower during the late autumn and winter in Belgium.
   Flower longevity on the plant.—About ten days; flower longevity is dependent on temperature; sepals persistent.
   Inflorescence height.—About 29.2 cm.
   Inflorescence diameter.—About 17.7 cm.
   Flower buds.—Length: About 2.1 cm. Diameter: About 1.3 cm. Shape: Elliptical to ovate. Texture and luster: Smooth, glabrous; matte. Color: Close to 145C to 145D; towards the apex, close to 145D; towards the base, flushed with close to 177B.
   Flower diameter.—About 6.6 cm.
   Flower depth (height).—About 2.7 cm.
   Petals.—Transformed into nectaries.
   Sepals.—Quantity and arrangement: Five, arranged in a single whorl. Length: About 3.8 cm. Width: About 2.3 cm. Shape: Broadly elliptical to broadly ovate. Aspect: Slightly concave to flat. Apex: Obtuse to broadly and bluntly acute. Base: Cuneate. Margin: Entire. Texture and luster, upper surface: Smooth, glabrous; matte. Texture and luster, lower surface: Smooth, glabrous; slightly glossy. Color: When opening, upper surface: Close to 155C; towards the base, close to 145B. When opening, lower surface: Close to 157D to lighter than 157D; towards the base, close to 195B; main vein occasionally slightly tinged with close to 176B. Fully opened, upper surface: Close to 145D; towards the base, close to 145B; color becoming closer to 138B with development. Fully opened, lower surface: Close to 157A to 157B; towards the base, close to 195B; main vein occasionally slightly tinged with close to 176B; color becoming closer to between 138B and 138C and veins, tinged with close to 178B with development.
   Peduncles.—Length: About 20.9 cm. Diameter: About 8 mm to 9 mm. Aspect: About 10° from vertical. Strength: Strong. Texture and luster: Smooth, glabrous; moderately glossy. Color: Close to 146D; fine and dense dots, close to 177B proximally flushed with close to 181A.
   Pedicels.—Length: About 3.2 cm. Diameter: About 2 mm. Aspect: About 35° from peduncle axis. Strength: Moderately strong. Texture and luster: Smooth, glabrous; moderately glossy. Color: Close to 151A to 151B; fine and dense dots, close to 178B.
   Reproductive organs.—Stamens: Quantity per flower: About 120. Filament length: About 1.4 cm. Filament color: Close to NN155D. Anther shape: Double reniform; basifixed. Anther size: About 1.5 mm by 1.5 mm. Anther color: Close to 154C. Pollen amount: Moderate. Pollen color: Close to 155A. Pistils: Quantity per flower: About five to seven. Pistil length: About 1 cm. Stigma diameter: About 0.3 mm. Stigma shape: Club-shaped. Stigma color: Close to 150C. Style length: About 9.5 mm. Style color: Close to 150D. Ovary color: Close to 145C. Nectaries (transformed petals): Quantity per flower: About 13. Length: About 9 mm. Diameter: About 2 mm. Shape: Tubular, slightly flattened. Apex: Obtuse. Texture and luster, inner and outer surfaces: Smooth, glabrous; slightly glossy. Color, immature, inner and outer surfaces: Close to 144C; proximally, close to 152D. Color, mature, inner and outer surfaces: Close to 144C; distally, close to 150B; proximally, close to 152D; with development, close to 144A; distally, close to 151D; proximally, close to 152D.
   Seeds and fruits.—Seed and fruit development have not been observed on plants of the new Helleborus to date.
Garden performance: Plants of the new Helleborus have been observed to have good garden performance and to tolerate rain, wind and high temperatures about 35° C. and to be suitable for USDA Hardiness Zones 5 to 9.
Pathogen & pest resistance: Plants of the new Helleborus have not been observed to be resistant to pathogens and pests common to Helleborus plants.

It is claimed:

1. A new and distinct Helleborus plant named ‘JHE00202’ as illustrated and described.
